(19) United States
(12) Patent Application Publication
(10) Pub. No.: US 2019/0358632 A1
(43) Pub. Date: Nov. 28, 2019

(54) Near Infrared Spectroscopy of Culture Media in Micro-Physiological Systems

(71) Applicant: Nirrin Bioprocess Analytics, Inc., Billerica, MA (US)

(72) Inventors: Bryan A. Hassell, Jamaica Plain, MA (US); John C. Ho, Sudbury, MA (US)

(21) Appl. No.: 16/419,690

(22) Filed: May 22, 2019

Related U.S. Application Data
(60) Provisional application No. 62/674,984, filed on May 22, 2018.

(51) Int. Cl. B01L 3/00; C12M 1/12; C12M 1/34

(52) U.S. Cl. CPC B01L 3/502715; C12M 41/46; C12M 25/04

(57) ABSTRACT

A culture medium of a micro-physiological (MP) system is analyzed by near infrared (NIR) absorption spectroscopy. The MP system can be microfluidic or a well-plate MP system. Spectra are obtained in real time and are analyzed to provide information such as, for example, the concentration of one or more analytes as a function of time.

### RELATED APPLICATIONS

This application claims the benefit under 35 USC 119(e) of U.S. Provisional Application No. 62/674,984, filed on May 22, 2018, which is incorporated herein by reference in its entirety.

### BACKGROUND OF THE INVENTION

Many applications in biology, medicine, pharmaceutical research and other areas rely upon information regarding the in vivo behavior of drugs. Developing new drugs for human medical indications, for example, has traditionally involved animal models. Due, for instance, to non-human genetics, however, animal studies can produce erroneous, false positive results, ultimately ending in failed clinical trials.

Micro-Physiological systems (MP systems) represent the next-generation of pre-clinical cellular models for drug discovery and development, with the goal of replacing drug efficacy and/or toxicity testing in animal studies.

Thought to be capable of recapitulating human organ level physiology and pathophysiology, MP systems are tissue models containing more complex microenvironments than previous human testing platforms (e.g., cells in a dish). To date, the term “MP systems” may encompass several technologies such as Organ-on-Chips, vascularized microfluidics, organoids, tumor spheroids, hybrid explant models, or novel microplate architectures, to name a few.

Integrated Organ-on-Chip MP representations of living organs and support structures for such systems are described, for example, by J. P. Wikswo et al. in International Publication No. WO 2013/086486 A1, also published as U.S. Pat. Appl. Pub. No. 2015/0004077 A1, both documents being incorporated herein by this reference in their entirety.

An organomimetic device involving a microfluidic device that can be used to culture cells in its microfluidic channels is described by J. Fernandez-Alcon in WO 2015/138032 (A2), also published as U.S. Pat. Appl. Pub. Nos. US 2016/326477 A1 and US 2017/327781 A1. As described in these documents, incorporated herein in their entirety by this reference, the organomimetic device can be part of dynamic system that can apply mechanical forces to the cells by modulating the microfluidic device and the flow of fluid through the microfluidic channels. The membrane in the organomimetic device can be modulated mechanically via pneumatic means and/or mechanical means. The organomimetic device can be manufactured by the fabrication of individual components separately, for example, as individual layers that can be subsequently laminated together. The device can include a thin and transparent portion disposed above or below central microchannels to allow non-invasive external observation of cellular activities using a microscope and various microscopy techniques such as surface plasmon resonance (SPR) spectroscopy.

Other documents pertaining to MP systems such as Organ-on-Chip include: Low, L. A. and Tagle, D. A. “Microphysiological Systems (‘Organs-on-Chips’) for Drug Efficacy and Toxicity Testing”, Clin. Transl. Sci. 10, 237-239 (2017); Benam, K. H. et al. “Engineered In Vitro Disease Models”, Annu. Rev. Pathol. Mech. Dis. 10, 195-262 (2015); and Hassell B. A. et al. “Human Organ Chip Models Recapitulate Orthotopic Lung Cancer Growth, Therapeutic Responses, and Tumor Dormancy In Vitro”, Cell Rep. 21(2), 508-516 (2017).

### SUMMARY OF THE INVENTION

Traditionally, molecules and cell secretions within MP systems (MPSs) are analyzed off-line, after removing a sample from the system. The sample can be immediately processed, e.g., on-site, frozen for future processing or sent to a facility. Because of the nature of the assays employed (ELISA (enzyme-linked immunosorbent assay), mass spectrometry, HPLC (high performance liquid chromatography), etc.) the analysis, even when conducted immediately, is not in real-time; acquiring the data can take hours to days. Thus, a mandatory time lag is imposed between a system stimulus (cytokines, small molecules, endotoxin, etc.) and observable system read-outs (cellular secretory response). This time lag limits the knowledge and understanding of biological systems, signaling, therapeutic responses, and so forth, to the resolution at which the sample is obtained from the system and the performance of the assay. Moreover, even when relatively higher resolution sampling may be possible, most assays are complex, intricate procedures, requiring multiple steps and hours of undivided attention. Any one of the required steps that is performed incorrectly will invalidate the results.

Therefore, a need exists for developing techniques to monitor MP system cultures in a non-invasive, non-destructive, real-time manner. A need also exists for approaches that are essentially hands-off and user-friendly.

The present invention relates to the use of near infrared (NIR) spectroscopy for detecting the presence and often the concentration of specific analytes within the culture medium of Micro-Physiological (MP) systems. Various types of MP systems can be used, including microfluidic MP systems, well-plate type MP systems and others. In one example, the MP system is a perfused organ on a chip device. Aspects of the invention can be applied to detecting the presence of analytes, for instance biologically secreted molecules in the culture medium and, in many cases, measuring the concentration of these analytes. In specific implementations, changes in analyte concentrations with time are monitored as well.

A sample of the culture medium of a MP system is introduced in a NIR sample probe that is positioned in the pathway of NIR electromagnetic radiation, between a source of NIR radiation and a detector. The NIR sample probe can be a static or a flow through cell. In some applications, with well-plate type MP systems, for instance, the NIR sample probe can go through or maybe into the MP system.

The invention addresses many of the problems associated with conventional approaches and can provide fast, real-time scans in a non-invasive, non-destructive manner. For example, techniques described herein can be conducted in real time, providing real-time data, with readouts capable of capturing behavior or phenomena occurring on time scales as diverse as seconds, minutes or hours, depending on the specifics being monitored. In some of its non-destructive approaches, the invention relies on culture media that can flow through the NIR interrogation probe and either return to the culture or be directly discarded. No freezing or storage for later analysis is required. In contrast to the intricate and complex assay work associated with conventional methods, the invention can be practiced with minimal and often without any sample preparation or calibration.

Versatile and adaptable, biomonitoring embodiments described herein can be applied to studying solutions for most or all types of MP systems.

While according to conventional thinking one may have considered NIR to lack the required sensitivity, it was found and demonstrated that use of a NIR spectrometer and a static cuvette-based probing system could detect TNF-alpha across a wide range of levels, e.g., picomoles (pM) to micromoles (μM).

In general, according to one aspect, the invention features a system for studying analytes in a culture medium of a micro-physiological (MP) unit. The system comprises a sample probe for containing a culture medium of the MP unit, wherein the sample probe is disposed in a pathway of near infrared (NIR) electromagnetic radiation. The system further includes a computer unit for analyzing NIR absorption spectra of analytes in the culture medium and reporting data.

In embodiments, the computer unit includes a chemometric model for reporting analyte concentrations as a function of time. Further, the MP unit might be a microfluidic MP unit or perfused, or a plate-well.

Further, the sample probe might be a static cell, a flow-through cell and/or an in-situ sample probe.

Another long-held belief that prevented NIR applications for aqueous media pertained to the problems raised by water absorption. In spite of the traditional view that water signal would obscure signal from analytes, it was found that using current instrumentation, improved detection techniques and/or numerical methods could yield valuable information, e.g., concentrations or concentration profiles as a function of time, of certain analytes.

In general, according to another aspect, the invention features a method for analyzing a culture medium of a micro-physiological (MP) unit. The method comprises directing near infrared (NIR) electromagnetic radiation to a NIR sample probe containing a sample of the culture medium, obtaining NIR absorption spectra of the sample of the culture medium, and analyzing the absorption spectra to obtain analyte concentration information.

In general, according to another aspect, the invention features a method for monitoring a culture medium of a micro-physiological (MP) unit, the method comprising directing near infrared (NIR) electromagnetic radiation to a NIR sample probe containing a sample of the culture medium, obtaining NIR absorption spectra of the sample of the culture medium as a function of time, and analyzing the absorption spectra to obtain a concentration profile of one or more analytes in the culture medium as a function of time.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic view showing arrangements A, B and C for interrogating a MP system culture sample using a NIR sample probe.

FIG. 2 is a block diagram of arrangements for interrogating MP systems using NIR spectroscopy.

FIG. 3 is a schematic block diagram of a configuration for a static, NIR sample probe with sample input and data readouts.

FIG. 4 is a schematic block diagram of a configuration of a flow-based, NIR sample probe with sample input and data readouts.

FIG. 5 is a schematic block diagram of a configuration of a static, in-situ style measurement, with sample input and data readouts.

FIG. 6 is a plot of predicted concentration versus measured concentration of TNF-alpha in an aqueous medium.

FIG. 7 is a series of concentration versus time plots comparing the prediction of a chemometric model applied to spectroscopic data for TNF-alpha to that measured offline with LPS stimulus.

FIG. 8 is a diagram showing an experimental approach in which IFN-gamma secreted by peripheral blood mononuclear cells is measured by practicing aspects of the invention and by ELISA.

FIG. 9 is a plot comparison of IFN-gamma concentrations determined over a time interval by ELISA and by techniques described herein.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

The term “multiple” refers to two or more elements or events.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Embodiments described herein rely on detection techniques based on near infrared (NIR) spectroscopy. Probing molecular overtone and combination vibrations, NIR spectroscopy covers the region of from 780 nanometer (nm) to 2500 nm of the electromagnetic spectrum. An overview of NIR spectroscopy can be found, for example, in an article by A. M. C. Davies in “An Introduction to Near Infrared (NIR) Spectroscopy”, http://www.impublications.com/content/introduction-near-infrarer-nir-spectroscopy. See also, Cervera, A. E., Petersen, N., Lantz, A. E., Larsen, A. & Gernaey, K. V. Application of near-infrared spectroscopy for monitoring and control of cell culture and fermentation. Biotechnol. Prog. 25, 1561-1581 (2009); and Roggo Y, et al., “A review of near infrared spectroscopy and chemometrics in pharmaceutical technologies”, Journal of Pharmaceutical and Biomedical Analysis, Volume 44, Issue 3, 2007.

Among its strength, NIR spectroscopy presents a non-invasive, non-destructive investigative approach, typically involving fast scan times. Instruments (spectrometers) for obtaining NIR spectra, often of bulk materials, are commercially available.

Nonetheless, conventional thinking has long considered NIR spectroscopy as lacking sensitivity. Presumably, this belief has prevented the application of NIR spectroscopy to the study of solutions, culture media in MP systems (also referred to herein as MP “units”).

Aspects of the present invention pertain to a method and system that rely on NIR spectroscopy to investigate MP systems, e.g., to detect, identify and/or quantify molecular species present in a MP culture medium.

In many cases, the medium is derived from three-dimensional (3-D) cell cultures, a more representative model of in-vivo environments than traditional 2-D cultures. In other cases, the cell culture, e.g., a 3-D cell culture, is combined with microfluidic technology, in a microfluidic device, for example.

Microfluidic devices are microscale structures that often involve automation and high throughput. Microfluidic chips, for instance, include a network of microchannels obtained by a suitable technique such as molding, engraving, soft-lithography, etc., that are connected to a macro-environment by holes of different dimensions, the latter defining pathways for injecting or evacuating fluids into and from the chip. Fluids can be manipulated to attain multiplexing, automation, and high-throughput systems. Techniques and equipment have been developed for managing fluids inside the microchannels and can include internal elements such as Quake valves, and other servo-based methods, or outer elements such as pressure controllers, pumps, and so forth.

In MP systems that include perfusion arrangements, the conduits (microchannels) and circulation of fluids typically mimic the in-vivo passage of fluid through the circulatory system or lymphatic system to an organ or a tissue, referring, e.g., to the delivery of blood to a capillary bed in tissue.

In one example, the MP system is an organ-on-chip, involving living cells on a scaffold of natural or synthetic material, resulting in 3-D structures that resemble human organs to a larger extent than the more traditional 2-D tissue samples. Considered a type of artificial organ, an organ-on-a-chip (OOC) is a multi-channel 3-D microfluidic cell culture chip that simulates mechanics and physiological responses and/or activities of entire organs and organ systems.

A lung-on-a-chip was reported by researchers from the Wyss Institute for Biologically Inspired Engineering at Harvard. See, e.g., Huh D. et al. “Reconstituting Organ-Level Function on a Chip”, Science 328, 1662 (2010). The small (channels are 400 μm (W)×100 μum (H)×12 mm (L)) device involved a porous membrane (with human lung cells on one side and human capillary blood cells on the other. Air flowed through a channel on the lung side and human blood cells flowed through a channel on the other. It was found that the arrangement was capable of stretching and relaxing, modeling breathing, and could mount an immune response to bacteria. Further applications of the lung model are described, for example, by Hassell B. A. et al. “Human Organ Chip Models Recapitulate Orthotopic Lung Cancer Growth, Therapeutic Responses, and Tumor Dormancy In Vitro”, Cell Rep. 21(2), 508-516 (2017).

Model organs such as kidney-on-a-chip, bone-marrow-on-a-chip, liver-on-a-chip, heart-on-a-chip, gut-on-a-chip, skin-on-a-chip, etc. also have been developed.

Other suitable MP systems include spheroids (or sphere cultures), another type of 3-D cell model that simulate a live cell's environmental conditions, in particular with respect to reactions between cells and those between cells and the matrix. Spheroids are often used to study changes in the physiological characteristics of cells, the difference in the structure of healthy cells and tumor cells, and the transformation of cells when forming a tumor.

Organoids, in-vitro derived 3-D cell aggregates, obtained from primary tissue or stem cells, also can be utilized. Organoids can be self-renewing and self-organizing and can exhibit organ functionality.

Introducing heterogeneity in organoids can result in 3-D hybrid arrangements that can include, for example, tumor cells and cells grown from normal tissue adjacent to the tumor.

Further implementations are based on plate-based microfluidic devices (utilizing a 12-, 24-, 96-, etc. wells, for instance). Such devices can be used as testing platforms for intact tumor tissues and can find application in identifying effective chemotherapeutic drugs for specific tumors and patients, to guide therapeutic decision-making.

The method and/or device described herein can be applied or adapted to other MP systems known in the art, or those developed in the future.

The culture medium from a MP unit such as described above, for instance, can be sampled in any suitable way, depending on factors including the particular type of experiment being carried out, specifics of the MP system and so forth.

In one embodiment, samples are removed from the MP culture and introduced (e.g., pipetted) into a static NIR sample probe, such as a suitable cuvette) for analysis.

In another embodiment, samples are perfused from the MP unit into a flow-cell type sample probe, with the sample passing (flowing) through the NIR beam for analysis. In many cases, the quality of signal, subsequent analysis and/or experimental repeatability will depend on the quality of the flow-cell. Thus, in specific implementations, the window through which the laser light is transmitted to interrogate the sample is made of a material that is optically transparent in the NIR spectral region. One nonlimiting example is quartz. Also desirable is having a path length that remains consistent from analysis to analysis. Custom microfluidic flow-cells are possible keeping such design factors under consideration. Other implementations utilize commercially available flow-cells such as those available from Hellma Analytics.

In further embodiments, samples are interrogated in situ, either through the sample if allowable or via the perfusion system.

The culture medium from a MP system, e.g., sampled as described above, is studied by NIR absorption spectroscopy, using radiation sources, dispersive elements such as diffraction gratings or prisms, detectors, filters, collimators, lenses, other optical components, etc., as known in the art.

In some cases, the NIR radiation source, dispersive components, optics, detector and so forth can be assembled in an arrangement tailored for a specific application, e.g., using suitable components. For example, one could use a source with associated grating or prism. In some situations, however, doing so may generate variability, a concern that can be addressed by relying on commercial NIR spectrometers (such as, for instance, those manufactured by Bruker, Foss, Axsun Technologies or other sources).

Some commercially available equipment (e.g., from Brucker or Foss) will also provide at least some additional optics (e.g., collimators, detectors, and/or lenses). In other cases (e.g., some Axsun Technologies systems), the apparatus will only include the laser engine. Proper launch optics (e.g., fiber optic cables, collimators, etc.) and collection optics (e.g., detectors, lenses, etc.) would have to be implemented. Since the way in which wavelength selection is achieved may be different for different instrument types, e.g. FT-NIR, diffraction gratings, or tunable cavity, determining which type to use may involve tradeoffs based on factors such as such as scanning resolution, speed, stability, etc.

Increased signal-to-noise ratios may be obtained using a specific system arrangement having: fiber optic out of the light generating system coupled to a collimator which collimates the beam to within the size of the photodetector employed; the sample (static or flow-cell or direct culture); and a detector. Other arrangements may place another lens and fiber optic after the sample; however, catching the light after the sample may, in some circumstances, involve losses.

Since air contains water molecules in amounts that fluctuate with the humidity in the room, fluctuating moisture levels can alter the signal and impact reproducibility. This can be addressed by reducing or minimizing the presence of air, for example, by reducing or minimizing the free space between collimator, sample and/or detector, by enclosing the experimental set-up in a dry or moisture-stable environment, and so forth.

Regardless of the type of MP system being studied, the critical part of the technique and/or arrangement described herein is having fluid in which the cell cultures are being maintained in the light path.

In one embodiment of the invention, the NIR sample probe is functionally the same in all situations. Accordingly, the NIR sample probe can be configured to work in conjunction with any microfluidic chip or microfluidic unit and is particularly well suited for arrangements that use perfusion.

The sample probe can be positioned before and/or after the microfluidic. Some implementations rely on interfacing the NIR probe with a suitable plate schematic (e.g., 12, 24, 96 well, etc.) where the medium could be sampled out of the culture into a probe interface. It may also be possible to have the NIR radiation shined through the sample in a configuration that places the detector on the opposite end of the plate, since these plates often are optically transparent.

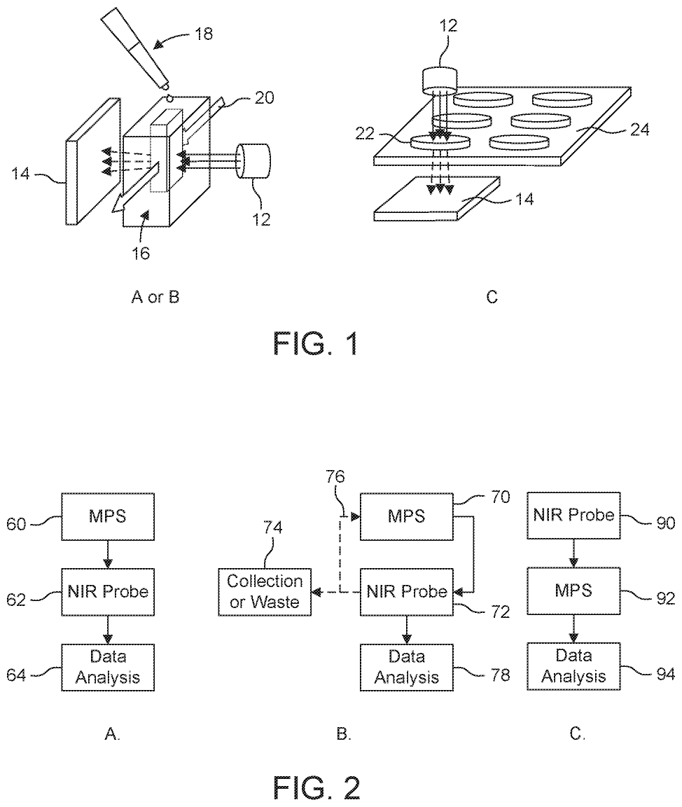

Shown in FIG. 1, for example, are arrangements A, B and C, generally including NIR radiation source 12 and NIR detector 14. The NIR radiation source may include light emitting diodes (LEDs), tungsten halogen lamps, micro electromechanical systems (MEMS)-based sources and infrared lasers. Spectral resolution may be dictated by grating-based solutions (scanning or fixed). Techniques employed include Fourier transform interferometry, filtering, acousto-optical tunable filters or Fabry-Perot tunable filters. Spectral sampling may be obtained by optical transmission, transflectance, reflection, scattering, or fluorescence. Suitable NIR techniques that can be used or adapted are described, for instance, in U.S. Pat. No. 9,540,701 B2.

Arrangements A and B utilize cuvette 16 for holding the sample. The cuvette sample probe can be designed to fit a particular support structure, such as found, for example, in the sample compartment of a spectrometer. The cuvette typically employs materials (such as quartz) that minimize absorbance of NIR radiation and can have any suitable dimensions, typically larger than the collimated spot size.

In the static arrangement A, the sample is extracted from the culture medium of the MP unit and transferred to the cuvette, using pipette 18, for example. This operation is often performed manually. Removing samples from a MP unit, introducing the sample into a NIR sample probe (cuvette) and/or extracting the sample from the sample probe also can be conducted automatically, by suitable robotics, for example.

In the flow-through arrangement B, the sample, perfused from an MP system, is fed and removed from the cuvette as shown by arrow 20.

Arrangement C employs an in-situ static configuration in which NIR radiation from NIR source 12 is directed through one well 20 of well-plate 22. Transmitted radiation reaches detector 14 disposed at the other side of the well-plate.

How the NIR probe is integrated with a specific type of MP system and data analysis is illustrated in FIG. 2. Arrangement A, for example, uses a static approach in which the sample is extracted from MP system 60 and introduced, using a pipette, for example, into NIR probe 62, which can be or can include a suitable cell or cuvette. Data analysis system (also referred to herein as “data analysis unit”) 64, e.g., a computer system provided with suitable hardware, software, interfaces, etc., for data gathering, data manipulation, data reporting, and so forth.

In arrangement B, the MP unit is interrogated with NIR spectroscopy in a flow-through configuration. In more details, a culture sample from MP system 70 is directed to the NIR probe 72 and effluent from the NIR probe is directed to waste collector 74, for disposal and/or reuse, as shown by arrow 76. Data is analyzed by data analysis system 78. For a given experiment, the flow-loop can be operated in a constant or continuous manner and data can be gathered as the sample goes in, through and out of the NIR sample probe.

Arrangement C includes NIR sample probe 90, MP system 92 and data analysis system 94. In a non-perfusion arrangement, a well-plate system, for instance, the NIR sample probe 90 can go through or even into the MP system.

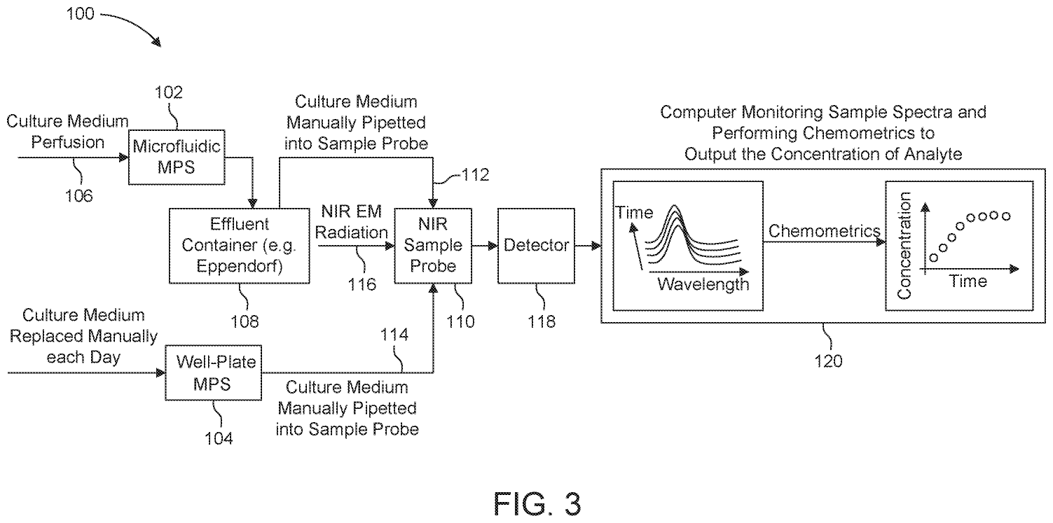

A static, a flow-through and an in-situ system are further described with reference to FIGS. 3, 4 and 5, respectively.

In FIG. 3, system 100 can employ microfluidic MP system 102 or well-plate MP system 104. In the former configuration, the culture medium circulates through perfusion arrangement 106 and effluent can be collected in effluent container 108, e.g., an Eppendorf tube. The sample can be extracted from the effluent container manually (using a pipette, for example) and introduced in NIR sample probe 110, as represented by arrow 112. As already noted, the sample handling operations can be automated.

In the alternative arrangement of well-plate MP system 104, culture medium can be added, e.g., daily or at another suitable time interval. A sample is extracted (pipetted, for instance) from the well-plate MP system and introduced, in manual or automated (e.g., robotic) fashion, into NIR sample probe 110, as shown by arrow 114.

NIR radiation 116 passes through NIR sample probe 110, containing culture medium from either the microfluidic MP 102 or well-plate MP system 104. The NIR electromagnetic radiation exiting the NIR sample probe is received by detector 118. From detector 118, the signal is handled by computer system 120 where NIR absorption spectra of the sample are monitored and manipulated to provide qualitative and/or quantitative information.

As shown in FIG. 3, for example, spectra can be obtained at suitable time intervals to observe changes in analyte concentration. NIR absorption spectra can be obtained at intervals ranging from every 0.1 minutes to once every minute, once every few minutes, hourly, or daily. Thus, the time resolution for monitoring complex behavior can be vastly improved compared to conventional techniques that require freezing and thawing and/or the complex and drawn-out protocols currently available. Chemical transformations of analyte molecules, protein aggregation, formation of postulated intermediates and/or products, post-translational modifications and so forth, also could be monitored.

In automated or semi-automated approaches, computer system 120 can also monitor and/or control experimental conditions, operational parameters and so forth. For instance, computer system 120 could be set up to control flow rates, valves, timings, etc. Controlling the MP system, e.g., as known in the art, also can be performed by a computer system other than computer system 120.

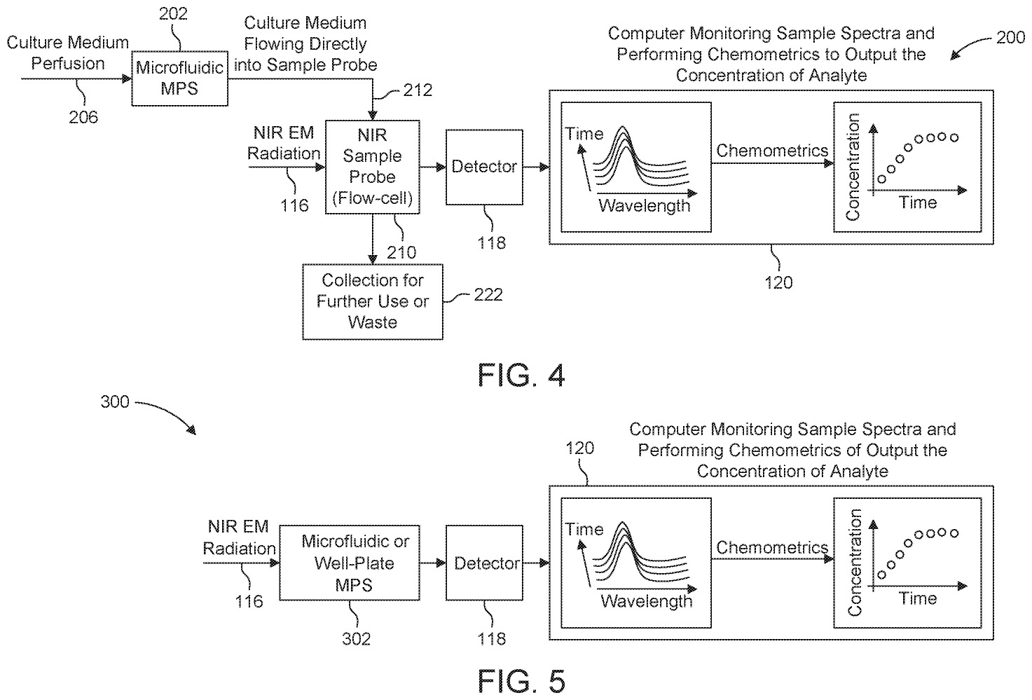

Shown in FIG. 4 is flow-through system 200, including microfluidic MP system 202, provided with perfusion arrangement 206. Culture medium from the MP system circulates to NIR sample probe 210, which is a flow-through cell, as shown by arrow 212. Effluent is collected in waste collector 222, for further handling. NIR sample probe 210 is in the pathway of NIR radiation 116. Signal from detector 118 is processed by computer system 120, essentially as described above.

MP systems such as 102 or 202 (FIG. 3 or 4, respectively) can be constantly perfused, as in the case of an organ-on-chip microfluidic, for instance. In such a case, it may be desirable to interrogate the culture medium downstream of the MP system, as shown in FIGS. 3 and 4.

In system 300 (FIG. 5), NIR radiation 116 passes through the culture medium in a microfluidic or well-plate MP system 302 and is detected by detector 118. Computer system 120 performs the data analysis, providing, for example, plots of analyte concentration as a function of time.

Many of the embodiments described herein provide multiplexing capabilities (measuring, typically simultaneously, several, often dozens or more analytes in a single run). If desired, it is also possible to measure a single analyte at a time.

As seen in FIGS. 1 through 5, the NIR sample probe is placed in the electromagnetic radiation pathway. Beyond this, the probe can be designed to function in the same way for all applications. Construction details may take into account static versus flow-through configurations and can provide for connections, conduits and/or circulation controls that allow the culture sample to flow in, through and out of the cell. Examples of specific probe designs can be found in some ASL-Analytical patents, such as, for example, U.S. Pat. No. 9,404,072 B2 which shows an interface probe specifically designed for disposable bioreactors, U.S. Pat. No. 9,146,189 B2 which shows an external cartridge which may connect to a bioreactor and also be disposable, or U.S. Pat. No. 9,360,422 B2 which shows an in-situ probe to be placed within a bioreactor and disposed afterwards.

Since many of the culture media of interest are aqueous, NIR bands caused by water molecule absorption (1440 nm and 1398 nm) can overlap or entirely obscure bands pertaining to the analyte of interest. Various approaches can be taken to correct for water NIR features. In U.S. Patent Application Pub. No. 20070211247, published on, Sep. 13, 2007, for example, Roumiana Tsenkova describes measuring the spectrum of a sample while exposing the sample to water-activating perturbations (WAP), thereby causing the response spectrum to change, and by detecting transitions of the response spectrum. Based on this, by performing spectral analysis and/or multivariate analysis, the components of the sample and/or the characteristics of the components can be determined. Other approaches that can be used to handle water effects in the NIR spectra obtained herein include but are not limited to probabilistic principle component analysis or Bayesian methods, novel calibration approaches, and/or novel segmentation of the spectrum to create unique analyte signatures.

Even without water-related spectral complications, the molecular overtone and combination bands characteristic of NIR often are broad, giving rise to complex spectra. The task of assigning specific spectral features to specific chemical species can be facilitated by methods such as, for example, multivariate (multiple variables) calibration techniques (e.g., principal components analysis, partial least squares, or artificial intelligence/neural networks).

Utilizing design of experiments (DOE), one may determine the optimal number of samples and sample ranges to build a robust calibration set. Building a good set of calibration spectra, e.g., with the application of multivariate calibration techniques, is critical to having predictive capabilities when analyzing water spectral measurements.

Specific implementations of the invention rely on chemometric techniques based, for instance, on mathematical and/or statistical methods for understanding the information obtained. NIR spectral patterns (band systems) of known analytes can serve to construct models that can be applied to future data. Various software packages have been developed and may be used or adapted to the requirements of the system and method described herein. Other software packages can be developed, using methods such as, for example: Principal Component Analysis (PCA), Regression (PLS, PCR, MLR, 3-way PLS) and Prediction, SIMCA (Soft Independent Modeling of Class Analogy), SIMCA and PLS-DA Classification, ANOVA and Response Surface Methodology, Multivariate Curve Resolution (MCR), Clustering (K-Means). In some cases, chemometrics could be performed to output the analyte concentration as a function of time on the basis of the NIR absorption spectra obtained sequentially in time.

Employing cloud storage can render the data accessible from different locations. Other features that can be used relate to a seamless data analysis approach where modeling, calculations, and/or reporting can be performed without a need for interpretation of the data by the user. Rather, the spectroscopic data (e.g., the absorption of electromagnetic NIR radiation by a culture medium) are interpreted and correlated to various analytes and their concentrations by the system.

In one embodiment, models of specific analytes are used to study cellular responses to perturbations. For instance, cells may be stimulated with LPS (lipopolysaccharides), other endotoxins, chemotherapeutics, other types of chemokines, and so forth. The immune response elicited can manifest, for example, by the production of lactate dehydrogenase (LDH), reactive oxygen species (ROS), cytokines or other immune-related factors, such as: tumor necrosis factor alpha (TNF-alpha), interleukins (e.g., IL-2, IL-8, etc.), interferons (e.g., IFN-gamma), colony stimulating factors (e.g., GM-CSF), chemokines (e.g., CXCL 12) and others. Chemometric models can be developed on example analytes. From these specific models, it is then possible to observe cellular response to perturbations in real-time and continuously (i.e., not needing to replace antibodies as in surface plasmon resonance).

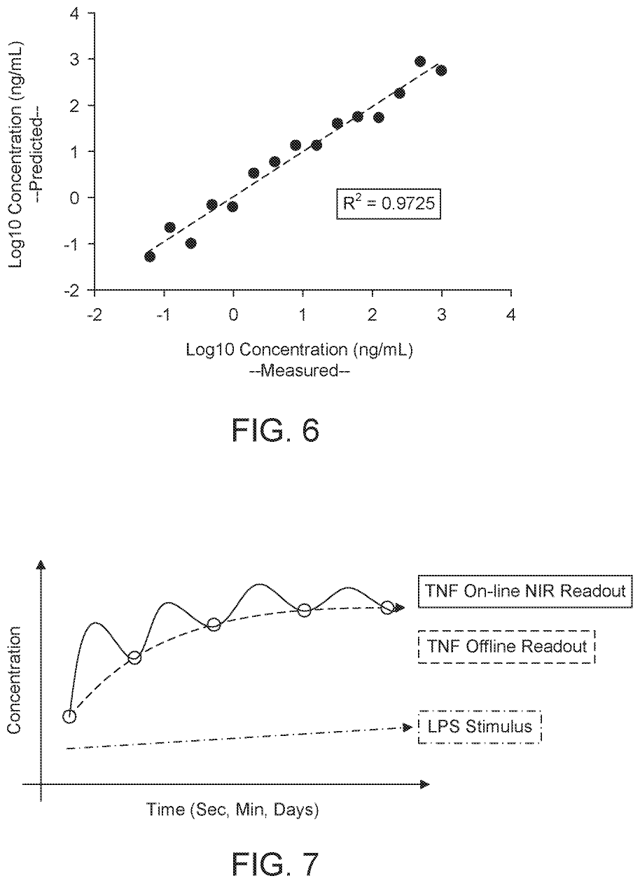

Shown in FIG. 6 is a plot of predicted versus detected levels of TNF-alpha across a wide range of concentrations (picomole (pM) to micromole (μM)) in an aqueous medium. The data was obtained by using NIR spectroscopy in a static, cuvette-based sample probe. FIG. 7 presents plots demonstrating the effects of a chemometric model applied to real-time spectroscopic data in an experiment measuring the response of TNF-alpha (upper curves) to endotoxin (lower curve). With a stimulus (LPS), one may take offline measurements (dashed circles) and from that assume that the cells secrete in some regular fashion. But there is evidence that responses are non-linear. Thus, with real-time monitoring, one can observe more complex behaviors (TNF on-line NIR readout). It is seen that when sampling off-line (circles) via an ELISA or other biochemical methods, there is an assumed trajectory and cellular response (dashed line). Applying chemometrics to real-time spectral data provides higher resolution measurements and thus enables the possibility of observing unique cellular responses, previously unknown (solid line).

Embodiments of the invention are further illustrated by the following non-limiting examples.

### Example—Monitoring PBMC Response to PMA and Ionomycin Stimulation

This example was conducted to assess the response of peripheral blood mononuclear cells (PBMC) to phorbol 12-myristate13 acetate (PMA) and ionomycin (I) stimulation. The generation (release) of IFN-gamma was monitored using a MPS probe such as described above and by ELISA, the current gold standard technique.

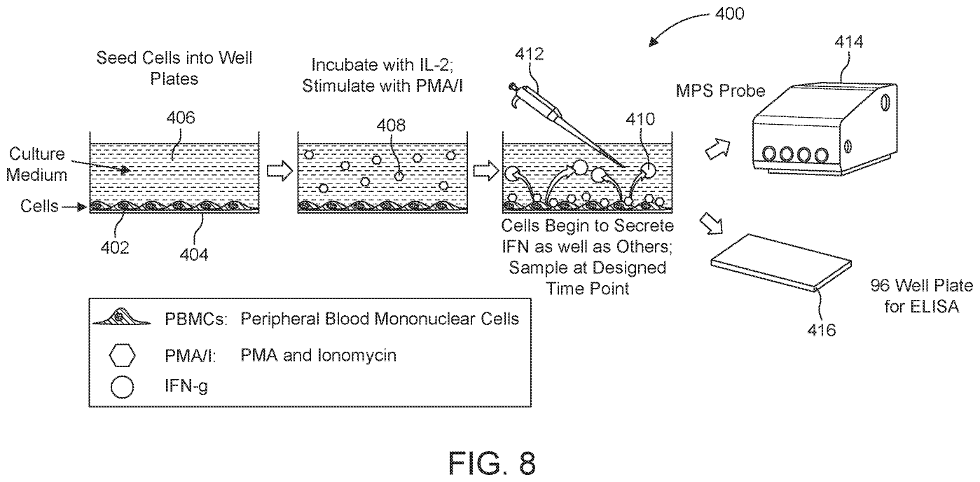

The protocol followed involved seeding cells into wells of 6-well plates and incubating the cells for 5 days in the presence of interleukin-2 (IL-2). At time t=0, the cells were stimulated with PMA/I. A schematic diagram of the experiment is presented in FIG. 8. In method 400, PBMC 402 are seeded into well plates 404 in the presence of a suitable culture medium 406. The cells are incubated with IL-2 and stimulated with PMA/I 408. Cells begin to secrete IFN-gamma 410 (and possibly other compounds). Sampling by a suitable technique 412, such as, for instance, pipette 18 in FIG. 1, arrangement A, is conducted at specified time points. Measurements are obtained using approach 414 according to embodiments of the invention and by ELISA, using well plate 416, e.g., a 96-well plate.

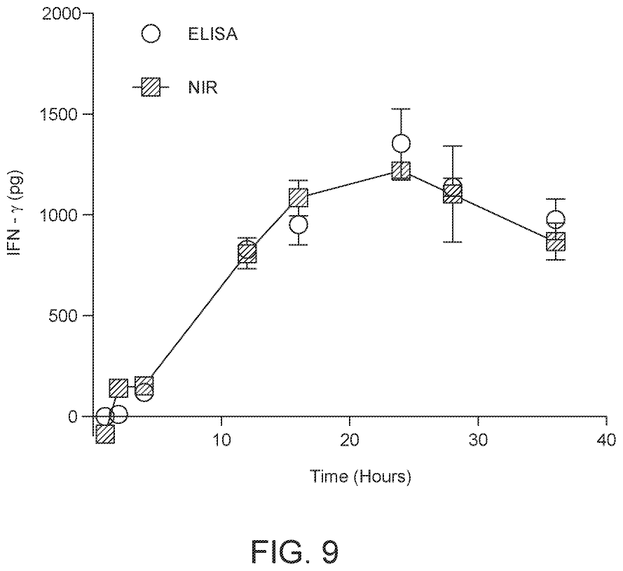

Two wells were used at each time point to build spectroscopic models for IFN-gamma prediction. More specifically, the data points in FIG. 9 represent 2 experimental replicates (samples were taken from 2 separate wells and used for ELISA).

The values obtained were used to build a chemometric model for the prediction of the other wells. Two additional wells per time point were used for scanning and predicting.

In more detail, sampling took place at designed time points, using 100 μL samples for ELISA (R&D Systems: Human IFN-gamma Quantikine ELISA Kit Catalog # DIF50) and 50 μL for scans conducted according to embodiments of the invention.

Shown in FIG. 9 are plots of the IFN-gamma concentrations in the wells as a function of time. The concentration profiles observed indicated that NIR based approaches described herein compared favorably with measurements obtained by ELISA, the current gold standard technique. Also, relative to ELISA, practicing embodiments of the invention allowed the use of smaller samples for reaching the same or comparable detection levels.

While this invention has been particularly shown and described with references to preferred embodiments thereof, it will be understood by those skilled in the art that various changes in form and details may be made therein without departing from the scope of the invention encompassed by the appended claims.

What is claimed is:

1. A system for studying analytes in a culture medium of a micro-physiological (MP) unit, the system comprising:
a sample probe for containing a culture medium of the MP unit, wherein the sample probe is disposed in a pathway of near infrared (NIR) electromagnetic radiation; and
a computer unit for analyzing NIR absorption spectra of analytes in the culture medium and reporting data.

2. The system of claim 1, wherein the computer unit includes a chemometric model for reporting analyte concentrations as a function of time.

3. The system of claim 1, wherein the MP unit is a microfluidic MP unit.

4. The system of claim 1, wherein the MP unit is perfused.

5. The system of claim 1, wherein the MP unit is a plate-well.

6. The system of claim 1, wherein the sample probe is a static cell.

7. The system of claim 1, wherein the sample probe is a flow-through cell.

8. The system of claim 1, wherein the sample probe is an in-situ sample probe.

9. A method for analyzing a culture medium of a micro-physiological (MP) unit, the method comprising:
directing near infrared (NIR) electromagnetic radiation to a NIR sample probe containing a sample of the culture medium;
obtaining NIR absorption spectra of the sample of the culture medium;
analyzing the absorption spectra to obtain analyte concentration information.

10. The method of claim 9, further comprising performing chemometrics to report analyte concentrations as a function of time.

11. The method of claim 9, wherein the MP unit is a microfluidic MP unit.

12. The method of claim 9, wherein the MP unit is perfused.

13. The method of claim 9, wherein the MP unit is a plate-well.

14. The method of claim 9, further comprising introducing the sample in the NIR sample probe.

15. The method of claim 9, wherein the NIR sample probe is a static cell.

16. The method of claim 9, wherein the NIR sample probe is a flow-through cell.

17. The method of claim 9, wherein the NIR sample probe is an in-situ sample probe.

18. A method for monitoring a culture medium of a micro-physiological (MP) unit, the method comprising:
directing near infrared (NIR) electromagnetic radiation to a NIR sample probe containing a sample of the culture medium;
obtaining NIR absorption spectra of the sample of the culture medium as a function of time;
analyzing the absorption spectra to obtain a concentration profile of one or more analytes in the culture medium as a function of time.

19. The method of claim 18, wherein the analyte is tumor necrosis factor alpha, an interleukin, an interferon, a colony stimulating factor, or a chemokine.

20. The method of claim 18, wherein the sample probe is a static cell.

21. The method of claim 18, wherein the sample probe is a flow-through cell.

22. The method of claim 18, wherein the sample probe is an in-situ sample probe.

23. The method of claim 18, wherein the MP unit is a microfluidic unit, or a well plate unit.
